(19) United States
(12) Patent Application Publication
(10) Pub. No.: US 2003/0103921 A1
(43) Pub. Date: Jun. 5, 2003

(54) Antiperspirant compositions comprising microemulsions

(75) Inventors: Richard Mark Brucks, Chicago, IL (US); Kathryn Elizabeth Gransden, Bebington (GB); Zhuning Ma, Chicago, IL (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/117,473

(22) Filed: Apr. 5, 2002

(30) Foreign Application Priority Data
Apr. 11, 2001 (GB) 0109143.8

(51) Int. Cl. A61K 7/34; A61K 7/38

(52) U.S. Cl. 424/66; 424/68

(57) ABSTRACT

Structured, antiperspirant microemulsions possibly in the form of liquid crystals, containing cosmetic oils, a solution of antiperspirant salt in a hydrophilic solvent, a surfactant and an oil structurant are provided. These microemulsions can be used in different types of Solid applicators such as soft solid and, particularly desirably, firm stick applicators. The structured microemulsions are preferably clear by virtue of a suitable choice of oil, solvent and structurant.

### FIELD OF THE INVENTION

This invention is related to microemulsions that contain cosmetically active ingredients. Particularly, this invention is related to antiperspirant salt-containing microemulsions and more particularly to microemulsions that are structured.

### BACKGROUND TO THE INVENTION

Microemulsions of the present invention contain a hydrophylic solvent, which most desirably is or comprises water. Microemulsions prior to being structured in the present invention are transparent or translucent, optically isotropic and thermodynamically stable mixtures of oil and water stabilized by surfactants and perhaps co-surfactants. The particle size of the dispersed droplets of a microemulsion is commonly about 100 to about 2000 angstroms, more preferably are about 100 to about 1000 angstroms. The droplets can form spontaneously or with a little energy input. Therefore, the microemulsions are simple to prepare and are not process dependent i.e. neither the order of addition of starting materials nor speed/type of mixing is particularly critical to the preparation of such microemulsions. It can be desirable to formulate antiperspirant compositions using non-structured microemulsions because microemulsions are easy and inexpensive to process and advantageously when they are formed, they can be inherently clear without requiring refractive index matching of the hydrophilic droplets and the cosmetic oils.

In a co-pending application, PCT/EP 00/0914, clear microemulsions are described which contain an active antiperspirant, water (which is a hydrophyilic solvent), a cosmetic oil, a nonionic surfactant and a cationic surfactant.

Many antiperspirant-containing microemulsion formulations are obtainable in liquid form, and these are suitable for application topically to human skin using pump or squeeze spray dispensers or via a roll on dispenser. However, not all consumers prefer to apply antiperspirant formulations in that manner. There are many consumers who like to employ a stick of antiperspirant which they rub across the local area of skin where they wish to control perspiration.

Microemulsions exist in the following forms: as water-in-oil, oil-in-water or as a bicontinuous phase, which is also called the surfactant phase. Bicontinuous phase microemulsions are found to solubilize a high amount of water and oil with lower levels of surfactant. The region around a bicontinuous phase microemulsion may transition into a swollen lamellar phase, otherwise known as a liquid crystal phase, and in certain cases these phases (microemulsion and liquid crystal) may co-exist. These phases exhibit birefringence, shear induced (streaming) birefringence, and are thixotropic, viscoelastic and transparent. Because some of these systems exhibit increased viscosity the technical literature may refer to them as microemulsion gels. However, in pratice, they do not contain a structurant, and thus to structure the cosmetic oils or for certain of the above-identified forms of microemulsions to increase the structure in the cosmetic oil, and particularly structure to a physical form which retains structural integrity when subjected to mild pressure, an additional constituent is needed.

As used herein, the term “structured microemulsion indicates water-in-oil or a bicontinuous phase, optionally together with a liquid crystal phase, or mixtures thereof containing a structurant.

Introduction of a structurant into a microemulsion not only alters the physical form of the product but also can alter its appearance. By suitable choice of structurant, it is possible to achieve a desired extent of opaqueness or translucency.

It is an object of the present invention to provide structured antiperspirant compositions, which contain high levels of antiperspirant salts, cosmetic oils and surfactants suitable for application to the axilla, further containing a structurant.

It is also an object of some embodiments of the present invention to provide structured antiperspirant compositions that do not require refractive index matching of the hydrophilic and hydrophobic phases in order to be clear.

It is also an alternative or additional object of at least some or other embodiments of the present invention to provide structured microemulsion antiperspirant compositions that require little energy to manufacture.

These and other objects of the present invention will become more readily apparent in the present application.

Patents and patent documents, which are cited in connection with the disclosed invention, are as follows:

DE 196 42 090 A1 discloses cosmetic or dermatologic compositions based on microemulsions.

U.S. Pat. No. 5,162,378 discloses water in oil microemulsions comprising cetyl dimethicone copolyol, water, silicone, alcohol, and 5-40% by weight of one or more salts.

U.S. Pat. No. 5,705,562 discloses a method of spontaneously forming a highly stable clear microemulsion by combining water, a volatile cyclic methyl siloxane or a volatile linear methyl siloxane and a silicone polyether surfactant. U.S Pat. No. 5,707,613 is in the same patent family as the just mentioned patent.

WO 94/22420 is concerned with silicone-based skin care products, which are applied to the skin as aerosols and form a clear gel on the skin.

WO 94/19000 discloses pharmaceutical compositions in the form of a microemulsion which comprises an oil, a mixture of high and low HLB surfactants in which the high HLB surfactant comprises an aliphatic, aryl or aliphatic-aryl sulfate or sulfosuccinate or salt thereof, an aqueous phase and a biologically active agent.

WO 94/08610 discloses pharmaceutical compositions in the form of microemulsions which comprise an oil, a mixture of high and low HLB surfactants in which the high HLB surfactant comprises a medium-chain fatty acid salt, an aqueous phase and a biologically active agent.

U.S. Pat. No. 5,575,990 discloses roll-on antiperspirant compositions which are clear and, when applied to the human skin, do not leave a visible white residue after drying. The clear antiperspirant roll-on compositions are stable under varying temperature conditions and provide a suitable cosmetically acceptable feel or sensation when applied to the human skin.

U.S. Pat. No. 5,487,887 discloses roll-on antiperspirant compositions and more particularly concerns antiperspirant compositions which are clear and stable under varying temperature conditions and, when applied to the human skin, do not leave a visible white residue after drying. The compositions in the form of an oil-in-water microemulsion, comprise an antiperspirant active 5-30, PEG-7-glyceryl cocoate 5-25, emollients 0.5-3, cyclomethicone 3-7, and water 53-60%.

None of the foregoing specifications exemplify antiperspirant microemulsions that are structured by the presence of a structurant.

### SUMMARY OF THE INVENTION

The invention relates to a composition in the form of a structured microemulsion comprising a solution of an antiperspirant salt in a hydrophilic solvent, a cosmetic oil, a surfactant and a structurant, the composition having a hardness of at least 0.003 N/mm².

### DETAILED DESCRIPTION OF THE INVENTION

The present invention is directed to antiperspirant microemulsions that are solidified.

Especially, the invention relates to structured microemulsions containing cosmetic oils, a solution of an antiperspirant salt in a hydrophilic solvent, a surfactant and a structurant. Preferably the constituents are so chosen that the resultant structured composition is at least translucent and stable.

In many embodiments, the microemulsions are primarily composed of bicontinous phase, but the compositions include water-in-oil, and microemulsion in liquid crystal form which are structured or further structured by the incorporation of a structurant. The microemulsions are novel antiperspirant compositions that can be used in stick types of applicators such as firm stick applicators or if suitably processed in soft solid applicators.

More specifically, the invention commonly relates to a composition in the form of a structured microemulsion comprising an antiperspirant salt, cosmetic oils, a nonionic surfactant and structurant.

In some preferred embodiments, the structured microemulsions in this invention contain inorganic salts such as antiperspirant salts and cosmetic oils and the solubilization of high levels of both employing in combination a quaternary ammonium surfactant and a nonionic surfactant.

The invention also relates to a method for controlling or preventing underarm perspiration and malodor, which comprises applying to the underarm area a composition according to the invention.

The characteristics of the structured microemulsions of this invention include one or more of:

The structured microemulsions exhibit stability over a relatively large range of temperature.

The structured microemulsions have a hardness of at least 0.003 N/mm².

The formulation is in the form of a firm or soft solid achieved by the incorporation of a structurant.

The types of the pre-structured microemulsions present are dependent on the ratio of aqueous constituents to the surfactant(s) and oil. The type relates in particular to the proportion of nonionic surfactant and the ratio of any quaternary surfactant thereto. When the percentage of the salt solution containing quaternary surfactant increases, the microemulsion changes from water-in-oil to oil-in-water type, and a bicontinuous microemulsion phase, or possibly a liquid crystal phase, will form in-between.

The microemulsions can contain a high level of inorganic salts.

The microemulsions contain a quaternary surfactant and a nonionic surfactant.

The microemulsions contain cosmetically acceptable oils.

A method for controlling or preventing underarm perspiration and malodor, which can be applied to the underarm area.

The application of the microemulsions can be accomplished by using solid or soft solid product dispensers.

As used herein % means weight percent based on the structured microemulsion unless otherwise specified.

As used herein the term cationic surfactant means quaternary ammonium surfactant.

The ingredients or starting materials set forth herein are either known or can be prepared according to known methods.

The compositions of the invention can be made by known methods or by methods that are analogous to known methods.

As used herein, microemulsions refer to stable microemulsions containing cosmetic oil; antiperspirant salts, a hydrophilic solvent like water, a structurant and a surfactant, commonly a nonionic surfactant. The microemulsions described herein which are structured by introduction of the structurant are primarily composed of a bicontinous phase but the compositions can include water-in-oil microemulsions. The compositions of the invention can also comprise a liquid crystal form. More specifically, the compositions of the invention before structurant addition are selected from the group consisting of a microemulsion, optionally in a liquid crystal form, or a mixture of a microemulsion and a liquid crystal. In some embodiments, the compositions of the invention comprise an antiperspirant salt, a cosmetic oil, structurant and a combination of at least one cationic quaternary surfactant and at least one nonionic surfactant.

The compositions of the invention are novel antiperspirant compositions that can be used in stick applicators.

All of the structured microemulsion compositions described herein contain antiperspirant salts and are stable over a large temperature range from room temperature to 45° C.-50° C., By stable herein is meant that the sticks retain their integrity at ambient temperature during storage.

The structured microemulsions herein usually have a hardness of at least 0.003 N/mm² at 25° C. Hardness, especially of soft solids, can be measured by a conventional sphere indentation technique, using a Stable Micro systems TA.XT2I™ Texture Analyser. In some formulations in the form of soft solids herein, the so measured hardness is up to 0.05 N/mm² at 25° C. and particularly up to 0.02 N/mm² at 25° C. In other and harder structured microemulsions in stick form, their hardness is greater than 0.05 N/mm² at 25° C. and particularly greater than 0.1 N/mm² at 25° C.

A description of the ingredients included in the compositions of the invention now follows.

Antiperspirant Salts

Antiperspirant salts contained in these microemulsions include, but are not limited to, aluminium chlorohydrate, aluminium dichlorohydrate, aluminium sesquichlorohydrate, aluminium chlorohydrex propylene glycol complex, aluminium dichlorohydrex propylene glycol complex, aluminium sesquichlorohydrex propylene glycol complex, aluminium chlorohydrex polyethylene glycol complex, aluminium dichlorohydrex polyethylene glycol complex, aluminium sesquichlorohydrex polyethylene glycol complex, aluminium zirconium trichlorohydrate, aluminium zirconium tetrachlorohydrate, aluminium zirconium pentachlorohydrate, aluminium zirconium octachlorohydrate, aluminium zirconium trichlorohydrex glycine complex, aluminium zirconium tetrachlorohydrex glycine complex, aluminium zirconium pentachlorohydrex glycine complex, aluminium zirconium octachlorohydrex glycine complex, aluminium chloride or buffered aluminium sulfate.

Antiperspirant actives for use herein are often selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Aluminium halohydrates are usually defined by the general formula Al₂(OH)ₓQ_{y} or a hydrate thereof in which Q represents chlorine, bromine or iodine, x is variable from 2 to 5 and x+y=6. The level of hydration is variable for example wherein there are up to about 6 or higher water molecules.

Zirconium actives can usually be represented by the empirical general formula: ZrO(OH)_{2n-nz}B_{z} or a hydrate thereof in which z is a variable in the range of from 0.9 to 2.0 so that the value 2n-nz is zero or positive, n is the valence of B, and B is selected from the group consisting of chloride, other halide, sulphate, sulfate and mixtures thereof. Possible hydration to a variable extent is represented by wH₂O. It is preferable that B represents chloride and the variable z lies in the range from 1.5 to 1.87. In practice, such zirconium salts are usually not employed by themselves, but as a component of a combined aluminium and zirconium-based antiperspirant. The level of hydration is variable for example wherein there are up to about 6 or higher water molecules.

The above aluminium and zirconium salts may have coordinated and/or bound water in various quantities and/or may be present as polymeric species, mixtures or complexes. In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine, which has the formula CH₂(NH₂)COOH.

Complexes of a combination of aluminium halohydrates and zirconium chlorohydrates with or without with amino acids such as glycine can be employed in this invention. Certain of those Al/Zr-glycine complexes are commonly called ZAG in the literature. Aluminium-Zirconium actives or ZAG actives generally contain aluminium, zirconium and chloride with an Al/Zr ratio in a range from 2 to 10, especially 2 to 6, an Al/Cl ratio from 2.1 to 0.9. ZAG actives also contain a variable amount of glycine. In certain conditions, salts with an Al/Zr ratio greater than 2 (also known as low-zirconium actives) may be preferred. Actives of these preferred types are available from Westwood, from Summit and from Reheis.

Other antiperspirant-salt actives that may be utilized include astringent titanium salts, for example those describe in GB 2299506A.

In some embodiments, it is preferable to employ aluminium chorohydrate. This can enable a wider choice of structurant to be employed for obtaining clear or at least translucent structured compositions.

The proportion of solid antiperspirant salt in a composition normally includes the weight of any water of hydration and any complexing agent that may also be present in the solid active. However, when the salt is in solution, its weight excludes any water present.

The antiperspirant active will often provide from 1 to 40% by weight of the product, especially up to 30% by weight and particularly from 5% to 26% of the product.

Hydrophilic Solvent

The hydrophilic solvent can comprise water or any water-miscible fluid. Preferably the solvent comprises at least 60% water, more preferably at least 75% water and particularly at least 90% water, % being by weight based on the total weight of solvent.

In addition to aluminium salts, the hydrophilic solvent in this invention, can solubilize monovalent, divalent and trivalent salts. The salts include sodium chloride, sodium sulfate, calcium chloride, calcium sulfate, magnesium chloride, aluminium sodium lactate, and mixtures thereof, if desired.

Other ingredients which can form a part of the solvent comprise a cosolvent, including liquid polyhydric alcohols, such as glycols, eg propylene glycol, dipropylene glycol or and the ether derived from propylene glycol and glycerol, or glycerol or mixtures thereof and yet other ingredients which can be dissolved in the hydrophilic solvent phase include buffers, solid glycols, sugars, cyclodextrins, preservatives, antimicrobials, fragrances, chelating agents, amino acids, antimicrobials, anticholinergics, water-soluble polymers etc.

Hydrophilic Solution Content

The antiperspirant salts or other ingredients in the hydrophilic solvent can be dissolved into the solvent first and then combined with the oils. Solvent content in the final formulations can range from 1% to 60%, 5% to 30% is preferred and 10% to 25% is the most preferred. The hydrophilic solution preferably comprises from 2 to 70%, more preferably from 10 to 60% and especially at least 15% by weight of the structured microemulsion.

Oil Constituents

The oil phase of the compositions of the invention can contain in addition to cosmetic oils such as water-immiscible oils having a melting point of not higher than 20° C., such fatty acid esters, fatty acid ethers, branched long chain alcohols or ethoxylated alcohols, hydrocarbons, fatty acids, and glycerides containing a fatty residue, which have a suitable melting point volatile or non-volatile silicone fluids at 20° C. Cholesterol and some other lipids can be incorporated with the oil phase to act as emollients. The oil phase concentration can range from 0% to 95%, but 20% to 60% is preferred.

Silicone fluids that may be included in compositions of the invention include volatile and non-volatile silicone fluids such as cyclomethicones and dimethicones. Volatile cyclo or linear methicones desirably contain 4, 5 or 6 silicon atoms. Examples of commercially available silicone oils include oils having grade designations 344, 345, 244, 245 and 246 from Dow Corning Corporation; Silicone 7207 and Silicone 7158 from Union Carbide Corporation; and SF1202 from General Electric.

The hydrophobic carrier employed in compositions herein can alternatively or additionally comprise non-volatile silicone oils, which include polyalkyl siloxanes, polyalkylaryl siloxanes and polyethersiloxane copolymers. Commercially available non-volatile silicone oils include products available under the trademarks Dow Corning 556 and Dow Corning 200 series or DC704. Incorporation of at least some non-volatile silicone oil having a high refractive index such as above 1.5, such as selected polyalkylarylsiloxanes can be advantageous. One suitable non-volatile silicone is phenyl tris(trimethylsiloxy)silane.

Fatty esters in the compositions of the invention herein are commonly selected from the group consisting of cetyl octanoate, C12-15 alcohol benzoate, isostearyl benzoate, diisopropyl adipate, isopropyl palmitate, isopropyl myristate and mixtures thereof.

Hydrocarbon oils such as aliphatic hydrocarbons (Permethyl 102A™, Permethyl 101™); hydrogenated polybutenes; hydrogenated polydecenes (Silkflo™); dioctylcyclohexane; mineral oil, cyclohexane and mixtures thereof may suitably be included in the compositions of the invention.

SurfactantS

Nonionic Surfactants

The compositions herein commonly comprise a non-ionic surfactant. The nonionic surfactant or co-surfactants employed in the compositions of the invention can be polyethoxylated alcohol ethers or esters, polyglycerol mono or di-esters, glyceryl esters or branched guerbet ethoxylates or alcohols, or long chain carboxylic acids or combinations thereof. These compounds have a hydrophilic-lipophilic balance (HLB) of between about 2 to about 15 and preferably less than about 12.

Various suitable nonionic surfactants herein comprise a polyoxyalkylene moiety, especially such a moiety containing from about 2 to 80, and especially 5 to 60 oxyethylene units, and/or contain a polyhydroxy compound such as glycerol or sorbitol or other alditols as hydrophilic moiety. As hydrophobic moiety, the nonionic surfacatants commonly contain an alkyl, alkenyl or aralkyl residue, normally containing from about 8 to 50 carbons and particularly from 10 to 30 carbons, of which alkyl is more often employed. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil sunflower seed oil or soya bean oil. Such non-ionic surfactants can also be derived from a polyhydroxy compound such as glycerol or sorbitol or other alditols. Non-limiting examples of such surfactants include Laureth-2 to Laureth 20, ceteareth-10 to ceteareth-25, ceteth-10 to ceteth-25, steareth-10 to steareth-25, and PEG-15- to PEG-25-stearate or distearate.

Non-limiting examples of glycerol-derived surfacatnats include polyglycerol-3 diisostearate; glycerol oleate; poly glycerol-2 monoisostearate; polyglycerol-2 diisostearate, glyceryl isostearate. The most preferred ones include polyglyceryl-3 diisosterate, glyceryl isostearate and glycerol oleate or combinations thereof.

Structured microemulsions herein often contain from 2 to 45%, sometimes up to 25% and particularly from 4 to 20% by weight nonionic surfactant. The formulations herein preferably comprise a quaternary ammonium surfactant.

Combinations of a cationic, quaternary ammonium surfactant(s) and a nonionic surfactant are more preferably employed in the compositions of the invention.

The quaternary surfactant in the invention formulations either increase the stability of the microemulsion before structurant is added by rendering it less sensitive to temperature or fluctuations in temperature, or simply permit the microemulsion to form. The preferred cationic surfactants employed in compositions of the invention are alkylamidopropyl alkyldimonium quaternaries.

The preferred cationic quaternary surfactants have the following structure:

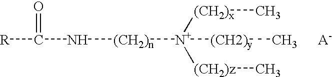

wherein

n is one to six.

x is zero to three

y is zero to three

z is zero to three

x, y and z can be the same or different,

with the proviso that x+y+z≦6

wherein R is a ricinoleic derivative:

CH₂—(CH₂)₅—CH(OH)—CH₂—CH—═CH—(CH₂)₇—

and wherein A⁻ is any physiologically acceptable counter ion which does not adversely affect the composition, and more specifically A⁻ can be selected from the group consisting of chloride, bromide, ethosulfate, methyl sulfate, lactate, acetate, nitrate or sulfate, or mixtures thereof variations on this structure, known to the art, can also be incorporated into embodiments of this invention. The variations on surfactant structure should exhibit solubility in the aqueous antiperspirant salt solution. If the above mentioned solubility is maintained then variations in the quaternary ammonium salts can include but are not limited to, increasing or decreasing the alkyl chain length, changing the position or removal of the hydroxyl group, changing the position or removing completely the double bond or combinations thereof.

The most preferred quaternary surfactant is ricinoleamidopropyl ethyldimonium ethosulfate a compound according to the formula above wherein n=3, x=1, y=0, z=0, A⁻=ethosulfate and:

R represents CH₂—(CH₂)₅—CH(OH)—CH₂—CH═CH—(C₂)₇

The surfactant described just above is also known, under the following trade names, as Surfactol Q4 from CasChem Inc., Lipoquat R from Lipo Chemicals or Mackernium DC-159 from McIntyre Chemical. Preferably the quaternary surfactant is supplied in a concentrated form (>90% active) with a low free amine content. This form is readily miscible with the aqueous antiperspirant-salt solution.

The quaternary surfactant(s) in the compositions of the invention range from 0.1% to 30%, where 2% to 15% is preferred.

The weight ratio of cationic surfactant to hydrophilic solution containing antiperspirant salt normally ranges from 30/70 to 4/96, the ratio range of from 10/90 to 5/95 being preferred. The ratio of hydrophilic solution including salts, water and cationic surfactant to nonionic surfactant is normally 90/10 to 70/30, and the ratio from 90/10 to 80/20 is preferred.

Structurant

In the compositions described herein, the structurant is normally selected from a fibre-forming structurant, a wax or a non-cross linked oil-soluble or oil-dispersible polymer. The structurant normally comprises from 3 to 25% by weight of the microemulsion.

In many embodiments herein, the eventual hardness of the structured microemulsion depends not only on the amount and type of structurant employed but also upon the processing conditions employed. The hardness can be varied by, for example, subjecting the composition to shear forces, particularly high shear forces, at or below the temperature at which the microemulsion is solidifying. For at least some of the formulations, this can lower the hardness.

One especially desirable class of structurants for use herein comprises the fibre forming structurants. Fibre forming structurants have the ability to first dissolve in the cosmetic oils at elevated temperature and upon cooling to below a solidifying temperature, form a solid phase within the oil, thereby structuring the oil, either to a soft or firm solid, so that the structured microemulsion has at least the minimum hardness specified hereinabove. Often such fibres are so thin that they are not visible to the unaided human eye.

Materials with this ability to gel hydrophobic organic liquids have been reviewed by Terech and Weiss in “Low Molecular Mass Gelators of Organic Liquids and the Properties of their Gels” Chem. Rev 97, 3133-3159 [1997] and by Terech in Chapter 8, “Low-molecular weight Organogelators” of the book “Specialist surfactants” edited by I D Robb, Blackie Academic Professional, 1997.

A number of fibre-forming structurants are identified below: U.S. Pat. No. 5,750,096 is one of several documents which teaches that gelation can be brought about using esters or amides of 12-hydroxy stearic acid. The alcohol used to form such an ester or the amine used to form such an amide may contain an aliphatic, cycloaliphatic or aromatic group with up to 22 carbons therein. If the group is aliphatic it preferably contains at least three carbon atoms. A cycloaliphatic group preferably contains at least five carbon atoms and may be a fixed ring system such as adamantyl.

Other fatty acids with C₈ or longer alkyl chains may he used and amides thereof can also be used. A specific example is lauric monoethanolamide also termed MEA lauramide:

N-acyl amino acid amides and esters are also known to structure liquids. We have established that they do so by forming fibrous networks. They are described in U.S. Pat. No. 3,969,087. N-Lauroyl-L-glutamic acid di-n-butylamide is commercially available from Ajinomoto under their designation GP-1.

Further materials which have been disclosed as gelling agents are the amide derivatives of di and tribasic carboxylic acids set forth in WO 98/27954 notably alkyl N,N′dialkyl succinamides.

Lanosterol, as disclosed in U.S. Pat. No. 5,635,165 may suitably be used if the water-immiscible liquid is silicone oil It is commercially available, e.g. from Croda Chemicals Ltd, and as supplied it contains some dihydrolanosterol. This impurity in the commercial material does not need to be removed.

A structurant which is the subject of WO 00/61096 is a combination of a sterol and a sterol ester. In its preferred form the sterol satisfies either of the two formulae:

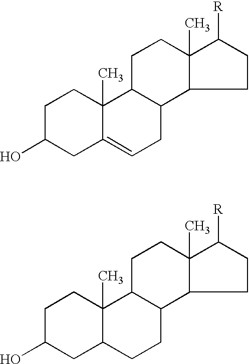

in which R represents an aliphatic, cycloaliphatic or aromatic group, and preferably a linear or branched aliphatic saturated or unsaturated hydrocarbon group. R desirably contains from 1 to 20 carbons and preferably from 4 to 14 carbons.

It is particularly suitable to employ β-sitosterol or campesterol or cholesterol, or a hydrogenated derivative thereof, such as dihydrocholesterol, or a mixture of two or more of them. An especially preferred sterol is β-sitosterol,

The preferred sterol ester is oryzanol, sometimes referred to as γ oryzanol which contains material satisfying the following formula:

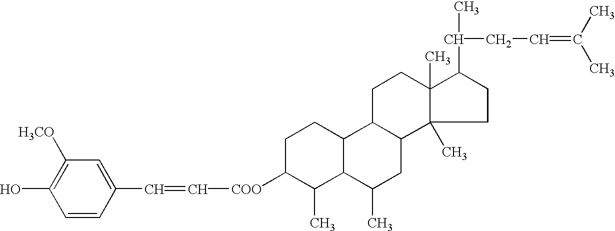

The sterol and sterol ester are used in a mole ratio that is normally selected in the range of from 10:1 to 1:10, especially from 6:1 to 1:4 and preferably in the range of from 3:1 to 1:2. Employment of the two system constituents within such a mole ratio range, and especially within the preferred range facilitates the co-stacking of the constituents and consequently facilitates the formation of a network that is readily able to structure the formulation.

Another structurant which is the subject of WO 00/61079 and which may be used in this invention is an ester of cellobiose and a fatty acid, preferably of 6 to 13 carbon atoms especially 8 to 10 carbon atoms. Preferably the 20 cellobiose is fully esterified, or nearly so, and is in the α-anomeric form to at least a substantial extent. In particular, in the context of cellobiose esterified with nonanoic acid, it is preferable that at least 70% and more preferable that at least 80% of molecules are esterified at the anomeric carbon in cellobiose. For convenience, such material is commonly referred to as cellobiose octanonanoate, even though a minor fraction of the material is in the form of a heptanonanoate ester. Likewise, it is preferable that at least 70% and preferably at least 80% of cellobiose nonanoate is in the α-anomeric form. The remainder of esterified cellobiose esters are in the β anomeric form.

The structure of such a compound, an esterified cellobiose, in its α-anomeric form is:

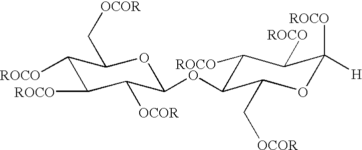

where R is an alkyl or alkenyl chain of 5 to 12 carbon atoms so that the acyl group contains 6 to 13 carbon atoms. Particularly preferred acyl groups incorporate a linear alkyl chain of 7 to 9 carbon atoms and are thus octanoyl, nonanoyl or decanoyl.

The acyl groups may have a mixture of chain lengths but it is preferred that they are similar in size and structure. Thus it is preferred that all of the acyl groups are aliphatic and at least 90% of the acyl groups have a chain length within a range such that the shorter and longer chain lengths in the range differ by no more than two carbon atoms, i.e. length in a range from m−1 to m+1 carbon atoms where m has a value in a range from 7 to 10.

Linear aliphatic acyl groups may be obtained from natural sources, in which case the number of carbon atoms in the acyl group is likely to be an even number or may be derived synthetically from petroleum as the raw material in which case both odd and even numbered chain lengths are available. Such materials and their preparation are described more fully in UP-A-6248312.

Although in a number of acylated sugars, the sugar nucleus is acylated by a single fatty acid, e.g. nonanoic acid, in some others, the acyl group at for example the anomeric carbon in the sugar nucleus can be substituted by a different acyl group from that on the other carbon atoms in that nucleus. In such compounds containing ester groups derived from a plurality of different carboxylic acids around the sugar nucleus, such as around the cellobiose nucleus, at least a fraction of the ester residue at the anomeric carbon is different from ester residues of the remaining sugar nucleus carbons. The ester group at the anomeric carbon contains a saturated or unsaturated, linear or branched chain hydrocarbon residue containing from 1 to 31 carbon atoms optionally substituted or an aromatic hydrocarbon residue, optionally substituted or a cycloaliphatic hydrocarbon, optionally substituted of which at least a fraction is different from the ester substituents on non-anomeric carbons, eg different from an octyl residue from esterification with nonanoic acid. At least a major fraction of such acylated sugars which are acylated by two different carboxylic acids are desirably in the form of the P anomer. Such materials and their method of preparation are described more fully in PCT/EP 01/10869.

It is especially desirable to employ the cellobiose ester, and particularly the cellobiose octaester in an oil mixture consisting essentially of hydrocarbon or silicone oils or a mixture thereof.

A further example of fibre forming structurant which is the subject of a co-pending application comprises an acylated maltose. Preferably, at least 7 of the available hydroxyl substituents of the maltose are acylated. It is desirable that β-anomer of the acylated maltose predominates over α-anomer. Thus the ratio of β-anomer to α-anomer is preferably from 65:35 to 100:0 and more preferably from 75:25 or 80:20 to 100:0.

It is envisaged that the acyl substituents of the maltose may be saturated or unsaturated, straight or branched chain hydrocarbon residue or possibly a cyclic aromatic or aliphatic hydrocarbon residue. They may incorporate substituent groups such as hydroxy or amino. A hydrocarbon chain may be interrupted by a heteroatom or a functional group containing a heteroatom such as an ether, ester or amide linkage.

The acyl substituents should contain at least 9 carbon atoms. It is unlikely that they will contain more than 22 carbon atoms. It is particularly preferred that each acyl group incorporates an alkyl or alkenyl chain R of 8 to 19 carbon atoms so that the acyl group contains 9 to 20 carbon atoms. Particularly preferred acyl groups incorporate a linear alkyl chain of 11 or 13 carbon atoms and are thus dodecanoyl or tetradecanoyl. Maltose esters described hereinabove can be prepared by methods analogous to those described in the aforementioned U.S. Pat. No. 6,248,312.

A further example of fibre forming structurant which is the subject of a co-pending application is a compound of the following general formula (TI):

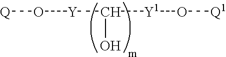

It is preferred that m is 2 so that the structurant compounds comply with a general formula (T2):

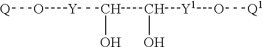

The groups Y and Y¹ will usually be identical, i.e. both methylene or both carbonyl. The groups Q and Q¹ may not be the same but often will be identical to each other.

If m is 2 and Y and Y¹ are methylene groups, the compound is a derivative of threitol, which is 1,2,3,4-tetrahydroxybutane, while if m is 2 and Y and Y¹ are carbonyl groups, the compound is a diester of tartartic acid, which is 2,3-dihydroxybutane-1,4-dioic acid.

It is preferred that each group Q and Q¹ contains an aromatic nucleus which may be phenyl or, less preferably, some other aromatic group. Thus, Q and Q¹ may be groups of the formula:

Ar—(CH₂)—

where Ar denotes an aromatic nucleus, notably phenyl or substituted phenyl and n is from 0 to 10.

An aromatic nucleus (Ar) is preferably unsubstituted or substituted with one or more substituents selected from alkyl, alkyloxy, hydroxy, halogen or nitro.

One substituent may be an alkyl or alkyloxy group with a long alkyl chain. Thus, a formula for preferred structurants of this invention can be given as (T3):

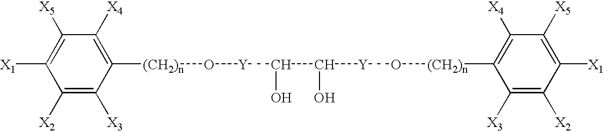

where

n=0 to 10, preferably 0 to 3, more preferably 1, 2 or 3;

Y=—CH₂— or >C═O

X=H, Cl, Br, F, OH, NO₂, O—R, or R, where R is an aliphatic hydrocarbon chain with 1 to 18 carbon atoms.

X₂ to X₅ are each independently H, Cl, Br, F, OH, NO₂, OCH₃, or CH₃

In these formulae above, the central carbon atoms which bear hydroxy groups are chiral centres. Thus, if m=2, Y and Y¹ are the same and Q and Q¹ are the same, the compounds will exist as R,R and S,S optically active forms as well as an optically inactive R,S form.

These compounds may be used as their optically active R,R or S,S forms or as a mixture of the two—which may be a racemic mixture.

A yet further class of amide structurants comprises 1,3,5-triamido-substituted cyclohexane (both —CO—NH—R′ and —NH—CO—R′). Such compounds and their preparation are described more fully in EP-A-1068854, in column 3, line 24 to column 4 line 47.

The amount of fibre forming structurant in the composition is often selected from the range of from 3 to 16%. In various preferred embodiments, the microemulsion is structured with a fibre-forming structurant as indicated herein in the presence of either no wax, or not more than a small amount, typically less than 3% and particularly less than 1% by weight.

Waxes

This term “wax” is conventionally applied to a variety of materials and mixtures which are melt at a temperature of 40° C. normally at not higher than about 95° C., and are water-insoluble and remain water-immiscible when heated above their melting point. Waxes form crystals in the water-immiscible oil when it cools from the heated state during processing, often in the form of needles or platelets.

Waxes are usually linear fatty alcohols, hydrocarbons, silicone polymers, esters of fatty acids or mixtures containing such compounds along with a minority (less than 50%) of other compounds. Naturally occurring waxes are often mixtures of compounds which include a substantial proportion likely to be a majority of fatty esters.

Linear fatty alcohols normally contain from 14 to 24 carbons, and known examples include cetyl and behenyl alcohol. The most commonly employed linear fatty alcohol is stearyl alcohol.

Examples of hydrocarbon waxes include paraffin wax, microcrystalline wax and polyethylenes waxes with molecular weight of 500 to 10,000, often 2000 to 10,000.

Examples of ester waxes include esters of C₁₆-C₂₂ fatty acids with glycerol or ethylene glycol and these may be made synthetically, often being di or triesters.

Examples of natural waxes include beeswax, carnauba and candelilla waxes which are of vegetable origin and mineral waxes from fossil remains other than petroleum. Montan wax, which is an example of mineral wax, includes non-glyceride esters of carboxylic acids, hydrocarbons and other constituents. Other suitable waxes include hydrogenated vegetable oils, including particularly hydrogenated castor oil, alternatively called castor wax.

Waxes useful in the present invention will generally be those found to thicken water-immiscible oils such as cyclomethicones when dissolved therein (by heating and cooling) at a concentration of 5 to 15% by weight.

The amount of wax in the composition is often selected from the range of from 3 to 20%.

Structured microemulsions using waxes often, though not always, produce an opaque product.

A master batch of an unstructured clear microemulsion can be structured using different structurants, for example either a wax or a fibre-forming structurant to produce as desired respectively an opaque and a clear product.

Non-Cross Linked Oil-Soluble Or Oil-Dispersible Polymer

One class of Non-Cross Linked Oil-Soluble or Oil-Dispersible polymer which has been found useful comprises the block copolymers of styrene with ethylene, propylene and/or -butylene available from Shell under their trade name KRATON G.

Conveniently the polymer can be employed in an amount of from 3 to 15% of the formulation.

Emollient

A material which can be employed herein in order to alter the sensory properties of the formulation comprises a mixture of linear cetyl, stearyl and myristyl alcohols, commonly in a weight ratio of 2:1:7, called an a wax. When employed in an amount for altering sensory properies, the a wax often has not been seen to structure the oil. There are some combinations of structurants ingredients which it is especially desirable to employ. One such combination comprises an emollient hydrocarbon wax such as an a wax with a fibre forming structurant and particularly an N-acyl amino acid amide such as N-Lauroyl-L-glutamic acid di-n-butylamide.

The following examples more fully illustrate embodiments of this invention, all percentages being by weight of the total formulation unless otherwise noted. The following specific examples, which are compositions of the invention, were made.

The Examples contained various of the following constituents:

Ingredients

| No | Chemical name | Trade name/Supplier |
|---|---|---|
| 1 | Cyclomethicone | DC245/Dow Corning |
| 2 | Polydecene | Silkflo 366NF/Lipo |
| | | Chemical INC |
| 3 | Cl2-Cl5 alcohol benzoate | Finsolv TN/Finetex |
| 4 | Iso stearyl alcohol | Witchol 66/Witco |
| 5 | Isopropylmyristate | Cognis |
| 6 | Aliphatic hydrocarbon | Permethyl 102A/Permethyl |
| | | Specialities |
| 7 | emollient | α-wax/own preparation |
| 8 | Cetyl octanoate | Trivent OC-16/Trivent |
| | | Chemical |
| 9 | Ricinoleamidopropyl | Surfactol Q4/CasChem |
| | ethyl dimonium | |
| | ethosulphate | |
| 10 | Laureth-4 | Brij 30/ICI |
| 11 | Ceteth-10 | Brij 56/ICI |
| 12 | Polyoxyethylene (20) | Brij 78/ICI |
| | stearyl ether | |
| 13 | Polyoxyethylene (20) | Arasolv 200/ICI |
| | Isohexadecyl ether | |
| 14 | Glycerol isosterate | Peceol Isostearique/ |
| | | Gattefosse |
| 15 | 43% aqueous ZAG (after | Westchlor Zr 41/Westwood |
| | dilution) | |
| 15a | 45% aqueous ZAG | Westchlor Zr 41/Westwood |
| 16 | Low zirconium aluminium | Low Zr penta (40%)/Reheis |
| | chlorohydrate | |
| 17 | 50% aqueous ACH | Westchlor 200/Westwood |
| 18 | Aluminium Zirconium | Rezal 67/Reheis |
| | penta chlorohydrate | |
| 19 | αCellobiose | own preparation |
| | octanonanoate | |
| 20 | αCellobiose | own preparation |
| | octadecanoate | |
| 21 | N-Lauroyl-L-glutamic | GP-1/Ajinomoto |
| | acid di-n-butylamide | |
| 22 | Stearyl alcohol | Lanette/Cognis |
| 23 | β-Sitosterol | Acros |
| 24 | γ oryzanol | Jan Dekker |
| 25 | polyglycerol-3- | Prisorine 3700/Uniquema |
| | diisostearate | |
| 26 | Glyceryl Oleate | Monomuls 90-018/Henkel |
| 27 | Deionised Water | local supply |
| 28 | Guerbet Ethoxylate | Novel II 18T-14/Condea |
| | | Vista |
| 29 | 2-octyldodecanol | Isofol/Condea Vista |
| 30 | Fragrance | |

When employed in the Tables listing the ingredients, oil and wax are self-explanatory, EML indicates an emollient, NS indicates a nonionic surfactant, QS a cationic surfactant and FFS a fibre-forming structurant. ZAG represents a Zirconium Aluminium Chlorohydrate complexed with glycine, ACH, Aluminium Chlorohydrate, and ZACH Zirconium Aluminium Chlorohydrate.

### EXAMPLE 1

The compositions in Example 1 were prepared according to the following procedure:

1. The oils were blended together, the structurants and nonionic surfactant introduced.

2. The oil mixture was then heated to a temperature of over 100° C. and mixing continued until the solids had dissolved an homogenous mobile fluid was obtained.

3. The mobile oil mixture was allowed to cool to below 100° C., but still above the temperature at which the oil phase set (observable in a preliminary test)

4. The cationic surfactant, if any, and other remaining water-miscible ingredients were mixed into the water and the aqueous solution heated to approximately the same temperature as that of the oil mixture obtained in step 3.

5. The oil mixture and the aqueous solution were mixed together thoroughly together until the mixture by eye comprised only a single phase, thus forming a microemulsion containing a structurant;

6. The resultant microemulsion of step 5 was poured into the barrel of a dispenser and allowed to cool to laboratory ambient temperature (about 23° C.).

The compositions and certain properties are summarised in Table 1 below. In Table 1, the hardness of the structured microemulsion was determine by determining the resistance to insertion of a blunt digital probe having a rounded end with a cross-section of about 1 cm². The clarity of the product was assessed by an experienced observer.

In the subsequent Tables, Clr indicates that the structured microemulsion was clear, Trl that it was translucent and Opq indicates that it was opaque.

TABLE 1

| | Example | | | | | | | | | |
|---|---|---|---|---|---|---|---|---|---|---|
| Ingredient | 1.1 | 1.2 | 1.3 | 1.4 | 1.5 | 1.6 | 1.7 | 1.8 | 1.9 | 1.10 |
| | % by weight | | | | | | | | | |
| 1 oil | 33.55 | 27.88 | 22.3 | 33.55 | 27.88 | 22.3 | 25.09 | 22.3 | 25.16 | 26.32 |
| 6 oil | 6.00 | 5.04 | 4.03 | 6.00 | 5.04 | 4.03 | 4.53 | 4.03 | 4.54 | — |
| 7 eml | 10.00 | 8.36 | 6.69 | 10.0 | 8.36 | 6.69 | 7.52 | 6.69 | 7.54 | — |
| 8 NS | 7.45 | 6.22 | 4.98 | 7.45 | 6.22 | 4.98 | 5.61 | 4.98 | 5.61 | — |
| 2 oil | — | — | — | — | — | — | — | — | — | 11.28 |
| 12 NS | 9.5 | 9.5 | 9.5 | 14.25 | 14.25 | 14.25 | 4.75 | 4.75 | 9.5 | — |
| 17 ACH | 28.5 | 38 | 47.5 | 23.75 | 33.25 | 42.75 | 47.5 | 52.25 | 42.85 | — |
| 16 ZACH | — | — | — | — | — | — | — | — | — | 39.95 |
| 14 NS | — | — | — | — | — | — | — | — | — | 9.4 |
| 9 QS | — | — | — | — | — | — | — | — | — | 7.05 |
| 23 FFS | 2.5 | 2.5 | 2.5 | 2.5 | 2.5 | 2.5 | 2.5 | 2.5 | 2.40 | 3 |
| 24 FFS | 2.5 | 2.5 | 2.5 | 2.5 | 2.5 | 2.5 | 2.5 | 2.5 | 2.40 | 3 |

Properties

| | | | | | | | | | |
|---|---|---|---|---|---|---|---|---|---|
| Hardness | firm | firm | firm | firm | soft | soft | soft | soft | |
| Clarity | Opq | Opq | Opq | Opq | Opq | Opq | Opq | Opq | Opq |

### EXAMPLE 2

In this Example, further examples of structured microemulsions were produced employing the same general method as in Example 1 and having similarly measured properties. The compositions and results are summarised in Table 2 below.

TABLE 2

| | Example | | | | | |
|---|---|---|---|---|---|---|
| Ingredient | 2.1 | 2.2 | 2.3 | 2.4 | 2.5 | 2.6 |
| | % by weight | | | | | |
| 1 oil | 27.88 | 22.3 | 22.3 | 25.09 | 22.3 | 23.48 |
| 6 oil | 5.04 | 4.03 | 4.03 | 4.53 | 4.03 | 4.24 |
| 7 eml | 8.36 | 6.69 | 6.69 | 7.52 | 6.69 | 7.04 |
| 8 NS | 6.22 | 4.98 | 4.98 | 5.61 | 4.98 | 5.24 |
| 12 NS | 9.5 | 9.5 | 14.25 | 4.75 | 4.75 | 5 |
| 17 ACH | 38 | 47.5 | 42.75 | 47.5 | 52.25 | 50 |
| 15 ZAG | — | — | — | — | — | — |
| 21 FFS | 5 | 5 | 5 | 5 | 5 | 5 |

Properties

| Hardness | Hard | Hard | Hard | Hard | Hard | Hard |
|---|---|---|---|---|---|---|
| Clarity | Trl | Trl | Trl | Trl | Trl | Trl |
| | Example | | | | | |
| Ingredient | 2.7 | 2.8 | 2.9 | 2.10 | 2.11 | 2.12 |
| | % by weight | | | | | |
| 1 oil | 27.95 | 22.36 | 22.36 | 25.16 | 22.36 | 21.90 |
| 3 oil | | | | | | 4.20 |
| 4 oil | | | | | | 8.00 |
| 6 oil | 5.05 | 4.04 | 4.04 | 4.54 | 4.04 | 4.00 |
| 7 eml | 8.38 | 6.70 | 6.70 | 7.55 | 6.70 | |
| 8 NS | 6.24 | 4.99 | 4.99 | 5.61 | 4.99 | |
| 11 NS | | | | | | 14.28 |
| 12 NS | 9.52 | 9.53 | 14.29 | 4.76 | 4.76 | |
| 15 ZAG | 38.1 | 47.62 | 42.86 | 47.62 | 52.39 | 42.86 |
| 21 FFS | 4.76 | 4.76 | 4.76 | 4.76 | 4.76 | 4.76 |

Properties

| | | | | | | |
|---|---|---|---|---|---|---|
| Hardness | Firm | Firm | Firm | Firm | Soft | Hard |
| Clarity | Opq | Opq | Opq | Trl | Trl | Opq |

### EXAMPLE 3

In this Example, further compositions were prepared and tested employing the general method and testing of Example 1. The compositions and results are summarised in Table 3 below.

TABLE 3

| | Example No | | |
|---|---|---|---|
| Ingredient | 3.1 | 3.2 | 3.3 |
| | % by weight | | |
| 1 oil | 20 | 12.5 | 20.4 |
| 6 oil | 20 | 12.5 | 3.69 |
| 7 wax | | | 6.12 |
| 8 NS | | | 4.56 |
| 10 NS | 7.5 | 10 | — |
| 13 NS | 7.5 | 10 | — |
| 12 NS | — | — | 11.5 |
| | 28 | 36 | |
| 27 Water | 7 | 9 | |
| 15 ZAG | — | — | 39 |
| 22 wax | 10 | 10 | 11.5 |

Properties

| | | | |
|---|---|---|---|
| Hardness | Firm | Firm | Soft |
| Clarity | Opq | Opq | Opq |

### EXAMPLE 4

The compositions in this Example were made by either the general method of Example 1 or by a modification in which the structurant was not introduced into the oil phase until the middle of step 5, i.e. when the oils and aqueous solution of antiperspirant had been mixed together.

TABLE 4

| | Example No | | | | | |
|---|---|---|---|---|---|---|
| Ingredient | 4.1 | 4.2 | 4.3 | 4.4 | 4.5 | 4.6 |
| | % by weight | | | | | |
| 1 oil | 26.6 | 26.6 | 19.95 | 19.95 | 13.3 | 13.3 |
| 2 oil | 11.4 | 11.4 | 8.55 | 8.55 | 5.7 | 5.7 |
| 17 ACH | 32.3 | 32.3 | 38.76 | 38.76 | 45.216 | 45.216 |
| 27 Water | 8.07 | 8.03 | 9.69 | 9.69 | 11.309 | 11.309 |
| 9 QS | 7.125 | 7.125 | 8.55 | 8.55 | 9.975 | 9.975 |
| 14 NS | 9.5 | 9.5 | 9.5 | 9.5 | 9.5 | 9.5 |
| 19 FFS | 5% | — | 5% | — | 5% | — |
| 20 FFS | — | 5% | — | 5% | — | 5% |

Properties

| Hardness | Hard | Hard | Hard | Hard | Hard | Hard |
|---|---|---|---|---|---|---|
| Clarity | ranging from Clear to Translucent | | | | | |
| | Example No | | | | | |
| Ingredient | 4.7 | 4.8 | 4.9 | 4.10 | 4.11 | 4.12 |
| | % by weight | | | | | |
| 1 oil | 25.2 | 25.2 | 18.9 | 18.9 | 12.6 | 12.6 |
| 2 oil | 10.8 | 10.8 | 8.1 | 8.1 | 5.4 | 5.4 |
| 17 ACH | 34 | 34 | 40.8 | 40.8 | 47.6 | 47.6 |
| 27 Water | 8.5 | 8.5 | 9.2 | 9.2 | 11.9 | 11.9 |
| 9 QS | 7.5 | 7.5 | 9 | 9 | 10.5 | 10.5 |
| 14 NS | 9.0 | 9.0 | 9.0 | 9.0 | 9.0 | 9.0 |
| 19 FF9 | 5% | — | 4% | — | 3% | — |
| 20 FFS | — | 5% | — | 4% | — | 3% |

Properties

| | | | | | | |
|---|---|---|---|---|---|---|
| Hardness | Hard | Hard | Hard | Hard | Hard | Hard |

| | |
|---|---|
| Clarity | Ranging from clear to translucent |

### EXAMPLE 5

In this Example, the structured microemulsions were made by the modified process of Example 4 in which the structurant was introduced into a pre-blended mixture of the hydrophilic solution and the oils.

TABLE 5

| | Example No | | | | |
|---|---|---|---|---|---|
| Ingredients | 5.1 | 5.2 | 5.3 | 5.4 | 5.5 |
| | % by weight | | | | |
| 1 oil | 10.39 | 20.55 | 28.16 | 10.74 | 15.2 |
| 2 oil | | | 10.91 | | |
| 6 oil | 24.26 | 41.36 | | 24.99 | 15.2 |
| 26 oil | | 6.94 | | | |
| 29 oil | | | | | |
| 30 fragrance | | | 0.95 | | |
| 16 ZACH | 41.27 | | | 41.17 | |
| 17 ACH | | 22.23 | | | 19.00 |
| 27 Water | | | | | 4.56 |
| 9 QS | 7.27 | 3.92 | | 7.25 | — |
| 25 NS | 11.81 | | | 10.85 | — |
| 10 NS | — | — | — | — | 18.24 |
| 13 NS | — | — | — | — | 22.8 |
| 28 NS | | | 1.53 | | |
| 19 FFS | 5 | 5 | 5 | 5 | 5 |

Properties

| | | | | | |
|---|---|---|---|---|---|
| Hardness | na | na | na | na | na |
| Clarity | Hazy | hazy | Hazy | Hazy | Hazy |

| | | Example No | | | |
|---|---|---|---|---|---|
| Ingredient | | 5.6 | 5.7 | 5.8 | 5.9 |
| | | % by weight | | | |
| | 1 oil | 18.06 | 11.40 | 20.05 | 14.81 |
| | 6 oil | 42.14 | 26.6 | 46.75 | 34.56 |
| | 16 ZACH | | | | 31.29 |
| | 17 ACH | 23.54 | 40.38 | 4.96 | |
| | 9 QS | 4.34 | 7.12 | 0.87 | 5.52 |
| | 25 NS | 0.19 | 9.5 | 22.37 | — |
| | 26 NS | 6.73 | — | — | 8.82 |
| | 19 FFS | 5 | 5 | 5 | 5 |

Properties

| | | | | |
|---|---|---|---|---|
| Hardness | na | na | na | na |
| Clarity | Clear | Clear | Clear | Clear |

Hardness of structured microemulsions herein can be measured by a Texture Analyser in which a blunt probe can be moved into or out from a sample at a controlled speed and at the same time the applied force is measured. The parameter which is determined as hardness is a function of the force and the projected area of indentation.

A specific test protocol uses a Stable Micro systems TA.XT2I™ Texture Analyser. A sample of composition is made by heating the ingredients, pouring into a container and allowing to cool as described above. The container is a 15 ml glass jar with a wide mouth. A metal sphere, of diameter 9.5 mm, is attached to the underside of the Texture Analyser's 5 kg load cell such that it can be used for indenting a sample placed beneath it on the base plate of the instrument.

After positioning the sample, the sphere position is adjusted until it is just above the sample surface. Texture Expert Exceeds software is used to generate the subsequent motion profile used in the test method. This profile initially moves the sphere into contact with the sample and then indents the sphere into the sample at an indentation speed of 0.05 mm/s for a distance of 7 mm. At this distance the direction of motion of the sphere is immediately reversed to withdraw the sphere from the sample at the same speed of 0.05 mm/s. During the course of the test, the data acquired are time(s), distance (mm) and force (N) and the data acquisition rate is 25 Hz.

The data associated with each test are manipulated using standard spreadsheet software and used to calculate the hardness, H, at a travelled distance of 4.76 mm after initial contact with the sample, using the following equation:

H=F/A

(H expressed in N.mm⁻², F in N and A in mm⁻²) where F is the load at the same travelled distance and A is the projected area of the indentation. This area can be calculated geometrically and is equal to the area of a diametral plane of the sphere, i.e. n×(4.76)² mm².

The foregoing description and Examples illustrate selected embodiments of the present invention. In the light thereof and his own knowledge, various modifications would be suggested to one skilled in the art, all of which are within the spirit and scope of this invention.

1. A composition which is selected from the group consisting of a microemulsion, a liquid crystal, or a mixture of a microemulsion and a liquid crystal which comprises an antiperspirant salt in solution in a hydrophylic solvent, a cosmetic oil, a surfactant and a structurant which structures the cosmetic oil, the composition having a hardness of at least 0.003 N/mm².

2. A composition in accordance with claim 1 characterised in that said antiperspirant salt is selected from the group consisting ot aluminium, zirconium and mixed aluminium/zirconium salts.

3. A composition in accordance with claim 1 or 2 characterized in that said antiperspirant salt is a zirconium salt complexed with aluminium salts having coordinated or bound water.

4. A composition in accordance with any preceding claim characterised in that said antiperspirant salt is present in the microemulsion at from about 1 to about 30%.

5. A composition in accordance with claim 4 characterised in that said antiperspirant salt is present in the emulsion at from 5% to 26%.

6. A composition in accordance with any preceding claim characterized in that the hydrophilic solvent comprises water.

7. A composition in accordance with any preceding claim characterized in that the solution in the hydrophilic solvent further comprises a buffer, a glycol, a sugar, a cyclodextrin, a preservative, an antimicrobial, a chelating agent, a water-soluble polymer, an anticholinergic, a monovalent salt, a divalent salt, a trivalent salt, fragrances or mixtures thereof in aqueous solution.

8. A composition in accordance with any preceding claim characterised in that said hydrophilic solution is present at about 1% to about 60%, more preferably at 5% to 30%, and most preferably at 10 to 25% of the microemulsion.

9. A composition in accordance with any preceding claim characterised in that the cosmetic oil is structured by a fibre-forming structurant, a wax or a non-cross linked oil-soluble or oil-dispersible polymer.

10. A composition in accordance with any preceding claim characterised in that the structurant comprises from 3 to 25% by weight of the microemulsion.

11. A composition in accordance with claim 9 or 10 characterised in that the fibre forming structurant is selected from 12-hydroxystearic acid or amide or ester derivatives of the acid group, N-acyl amino acid amides or esters, lanosterol, a combination of a sterol plus a sterol ester, cellobiose polyesterified with a fatty acid, maltose polyesterified with a fatty acid, or a bisamido cyclohexane.

12. A composition in accordance with claim 11 characterised in that the fibre forming structurant is 12-hydroxy stearic acid, N-Lauroyl-L-glutamic acid di-n-butylamide, a combination of P sitosterol and y oryzanol, or a hepta or octaester of cellobiose with a C8 to C12 aliphatic carboxylic acid, or a mixture of fibre forming structurants.

13. A composition in accordance with claim 12 characterised in that the structurant comprises cellobiose octanonanoate.

14. A composition in accordance with any of claims 11 to 13 characterised in that the content of fibre-forming structurant is from 3 to 16% by weight of the microemulsion.

15. A composition according to claim 9 or 10 characterised in that the wax comprises a fatty alcohol, a hydrocarbon wax, beeswax, a plant wax or hydrogenated plant oil or silicone wax or mixture of any two or more thereof, each having a wax melting point of at least 40° C. and preferably at least 50° C.

16. A composition in accordance with claim 15 characterised in that the wax comprises castor wax and/or stearyl alcohol.

17. A composition in accordance with claim 15 or 16 characterised in that the wax comprises from 3 to 20% by weight of the microemulsion.

18. A composition in accordance with any of claims 11 to 13 characterized in that the composition contains less than 1% by weight of a wax.

19. A composition in accordance with any of claims 9 to 17 characterised in that the structurant comprises the fibre forming structurant in an amount of from 3 to 10% and the wax in an amount of from 4 to 12%, % s being by weight of the microemulsion.

20. A composition in accordance with claim 9 or 10 characterized in that the non-cross linked oil-soluble or oil-dispersible polymer comprises a block copolymers of styrene with ethylene propylene and/or butylene.

21. A composition in accordance with any preceding claim characterised in that said cosmetic oil comprises esters, ethers, long chain alcohols or ethoxylated alcohols, hydrocarbons, fatty acids, monoglycerides, diglycerides triglycerides, fragrances and volatile or non-volatile silicone fluids, and cholesterol.

22. A composition in accordance with claim 21 characterised in that said cosmetic oil comprises a silicone fluid which in turn comprises a volatile and/or non-volatile silicone.

23. A composition in accordance with claim 21 or 22 characterised in that said non-volatile silicone is phenyl tris(trimethylsiloxy)silane.

24. A composition in accordance with claim 21 characterised in that said cosmetic oil comprises essentially a hydrocarbon oil or a silicone oil or a Mixture thereofand the structurant comprises a cellobiose fatty acid ester, preferably an octaester.

25. A composition in accordance with claim 21 characterised in that said esters are selected from the group consisting of cetyl octanoate, C12-15 alcohol benzoate, isostearyl benzoate, diisopropyl adipate and mixtures thereof.

26. A composition in accordance with claim 21 wherein said hydrocarbon fluids are selected from the group of aliphatic hydrocarbons; hydrogenated polydecenes; hydrogenated polybutenes; dioctylcyclohexane; mineral oil, cyclohexane and mixtures thereof.

27. A composition in accordance with any preceding claim characterised in that the surfactant comprises a non-ionic surfactant.

28. A composition in accordance with claim 27 characterised in that the content of non-ionic surfactant is from 2 to 45%, such as 4 to 20% by weight.

29. A composition in accordance with any preceding claim characterised by further comprising a cationic quaternary ammonium surfactant.

30. A composition in accordance with claim 27 characterised in that the cationic quaternary ammonium surfactant has the following structure:

31. 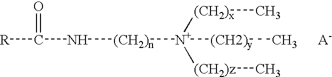
wherein
n is one to six.
x is zero to three
y is zero to three
z is zero to three
x, y and z can be the same or different
with the proviso that x+y+z≦6
wherein R is a ricinoleic derivative;
CH₂—(CH₂)₅—CH(OH)—CH₂—CH═CH—(CH₂)₇
wherein A⁻ is any physiologically acceptable counter ion which does not adversely affect the composition, and more specifically A⁻ can be selected from the group consisting of chloride, bromide, ethosulfate, methyl sulfate, lactate, acetate, nitrate or sulfate. or mixtures thereof.

32. A composition in accordance with claim 30 characterised in that in the formula n=3, x=l, y=0, z=0, A⁻ represents ethosulfate and R represents CH₃—(CH₂)₅—CH(OH)—CH₂—CH═CH—(CH₂)₇_{⁻}.

33. A composition in accordance with any preceding claim characterised in that said cationic quaternary ammonium surfactant is present at a concentration of from 0.1% to 30%, more preferably at 1% to 30%, and most preferably at 2% to 15%.

34. A composition in accordance with any preceding claim characterised in that the structured microemulsion has a hardness of greater than 0.1 N/mm² at 25° C.

35. A cosmetic method for controlling or preventing perspiration and malodor which method comprises applying, topically to skin an effective amount of a composition of claim 1.
